(19) United States
(12) Patent Application Publication
(10) Pub. No.: US 2011/0144741 A1
(43) Pub. Date: Jun. 16, 2011

(54) Coating Construct With Enhanced Interfacial Compatibility

(75) Inventors: Lothar W. Kleiner, Los Altos, CA (US); Jessica Renee DesNoyer, San Jose, CA (US); Syed Faiyaz Ahmed Hossainy, Hayward, CA (US)

(73) Assignee: Advanced Cardiovascular Systems, Inc., Santa Clara, CA (US)

(21) Appl. No.: 13/027,126

(22) Filed: Feb. 14, 2011

Related U.S. Application Data
(62) Division of application No. 11/453,419, filed on Jun. 14, 2006.

(51) Int. Cl. A61F 2/82; A61L 33/00; B32B 27/00

(52) U.S. Cl. 623/1.46; 427/2.3; 428/421

(57) ABSTRACT

The present invention provides a method of forming a coating on a medical device having a topcoat and a basecoat and an improved compatibility between a topcoat and a basecoat on the medical device.

### CROSS-REFERENCE TO RELATED APPLICATIONS

This application is a divisional of application Ser. No. 11/453,419, filed on Jun. 14, 2006, and published on Apr. 24, 2008 as U.S. Patent Application Publication No. 2008-0095918 A1, which is incorporated by reference in its entirety, including any drawings, herein.

### FIELD OF THE INVENTION

This invention is generally related to forming a coating having a construct with enhanced interfacial compatibility for implantable medical devices, such as drug delivery vascular stents.

### DESCRIPTION OF THE STATE OF THE ART

Stents are used not only as a mechanical intervention of vascular conditions but also as a vehicle for providing biological therapy. As a mechanical intervention, stents act as scaffoldings, functioning to physically hold open and, if desired, to expand the wall of the passageway. Typically, stents are capable of being compressed, so that they can be inserted through small vessels via catheters, and then expanded to a larger diameter once they are at the desired location. Examples in patent literature disclosing stents that have been applied in PTCA procedures include stents illustrated in U.S. Pat. No. 4,733,665 issued to Palmaz, U.S. Pat. No. 4,800,882 issued to Gianturco, and U.S. Pat. No. 4,886,062 issued to Wiktor.

Biological therapy can be achieved by medicating the stents. Medicated stents, e.g., stents with a coating that includes an agent, provide for the local administration of a therapeutic substance at the diseased site. In order to provide an effective concentration at the treated site, systemic administration of useful medication often produces adverse or toxic side effects for the patient. Local delivery is a preferred method of treatment in that smaller total levels of medication are administered in comparison to systemic dosages, but are concentrated at a specific site. Local delivery thus produces fewer side effects and achieves more favorable results.

Coatings on a medical device such as a stent are often desired to have a surface that can be modified to meet different biological or therapeutic needs. Sometimes, a topcoat including a pro-healing (PH) polymer can be coated on the surface of the device to facilitate recruiting of endothelial cells (ECs) (re-EC). Unfortunately, such a topcoat often has a poor interfacial compatibility with a hydrophobic layer of coating on a device (e.g., a coating of poly(vinylidene-co-hexapropene) (SOLEF®) (hereafter referred to as “base coat”). This leads to compromised mechanical and biological properties of the coating.

The embodiments described below address the above-identified problem.

### SUMMARY

Provided in the present invention is a method of forming a coating having a construct with an enhanced interfacial compatibility. The method comprising providing a co-solvent for the polymer for forming a basecoat and the polymer for topcoat, and forming the basecoat and the topcoat, respectively. The coating thus formed has an enhanced/improved interfacial compatibility and thus improved mechanical, physical and biological properties.

In some embodiments, interfacial compatibility between the topcoat and the basecoat can be improved by: (1) preparing or priming a substrate coating (basecoat) with a blank solvent spray, and (2) then spray-coating a topcoat formulation on the basecoat. In these embodiments, the solvent in the blank solvent spray is the solvent of the polymer in the basecoat. This method can result in an enhanced interfacial bonding and, thus, an enhanced interfacial compatibility even if a co-solvent for both the basecoat polymer and the topcoat polymer is difficult to find. As used herein, “a blank solvent” refers to a solvent having no polymer or agent dissolved therein.

In some embodiments, the topcoat can be formed by spray-coating a topcoat formulation on a basecoat in the presence of a solvent-rich atmosphere. The solvent is a solvent for the basecoat polymer and can plasticize or absorb into the basecoat. In these embodiments, the topcoat formulation solvent can be independent of the selection of the solvent for the basecoat polymer.

The coating described having the features described herein can include a bioactive agent. Some exemplary agents include, but are not limited to, paclitaxel, docetaxel, estradiol, nitric oxide donors, super oxide dismutases, super oxide dismutases mimics, 4-amino-2,2,6,6-tetramethylpiperidine-1-oxyl (4-amino-TEMPO), biolimus, tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N-1-tetrazolyl)-rapamycin (ABT-578), clobetasol, pimecrolimus, imatinib mesylate, midostaurin, prodrugs thereof, co-drugs thereof, or a combination thereof.

A medical device having the features described herein can be used to treat, prevent, or ameliorate a medical condition such as atherosclerosis, thrombosis, restenosis, hemorrhage, vascular dissection or perforation, vascular aneurysm, vulnerable plaque, chronic total occlusion, claudication, anastomotic proliferation (for vein and artificial grafts), bile duct obstruction, ureter obstruction, tumor obstruction, and combinations thereof.

### BRIEF DESCRIPTION OF THE DRAWINGS

FIG. 1 shows the endothelial cell coverage scores of PEA-TEMPO coated and PEA-TEMPO/everolimus coated stents as compared to bare metal stent (BMS), PBMA/SOLEF™ polymer, and PBMA/SOLEF™ coated stents.

FIG. 2 is the scanning electronic microscopy (SEM) image of the overview of PEA-TEMPO/PBMA/SOLEF™ (100 μg) coating system post-simulated use and ETO sterilization.

FIG. 3 is the scanning electronic microscopy (SEM) image of the OD overview of PEA-TEMPO/PBMA/SOLEF™ (100 μg) coating system post-simulated use and ETO sterilization.

FIG. 4 is the scanning electronic microscopy (SEM) image of the ID overview of PEA-TEMPO/PBMA/SOLEF™ (100 μg) coating system post-simulated use and ETO sterilization.

### DETAILED DESCRIPTION

Provided in the present invention is a method of forming a coating having a construct with an enhanced interfacial compatibility. The method comprising providing a co-solvent for the polymer for forming a basecoat and the polymer for topcoat, and forming the basecoat and the topcoat, respectively. The coating thus formed has an enhanced/improved interfacial compatibility and thus improved mechanical, physical and biological properties.

In some embodiments, interfacial compatibility between the topcoat and the basecoat can be improved by: (1) preparing or priming a substrate coating (basecoat) with a blank solvent spray, and (2) then spray-coating a topcoat formulation on the basecoat. In these embodiments, the solvent in the blank solvent spray is the solvent of the polymer in the basecoat. This method can result in an enhanced interfacial bonding and, thus, an enhanced interfacial compatibility even if a co-solvent for both the basecoat polymer and the topcoat polymer is difficult to find.

In some embodiments, the topcoat can be formed by spray-coating a topcoat formulation on a basecoat in the presence of a solvent-rich atmosphere. The solvent is a solvent for the basecoat polymer and can plasticize and absorb into the basecoat. In these embodiments, the topcoat formulation solvent can be independent of the selection of the solvent for the basecoat polymer.

The coating described having the features described herein can include a bioactive agent. Some exemplary agents include, but are not limited to, paclitaxel, docetaxel, estradiol, nitric oxide donors, super oxide dismutases, super oxide dismutases mimics, 4-amino-2,2,6,6-tetramethylpiperidine-1-oxyl (4-amino-TEMPO), biolimus, tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N-1-tetrazolyl)-rapamycin (ABT-578), clobetasol, pimecrolimus, imatinib mesylate, midostaurin, prodrugs thereof, co-drugs thereof, or a combination thereof.

A medical device having the features described herein can be used to treat, prevent, or ameliorate a medical condition such as atherosclerosis, thrombosis, restenosis, hemorrhage, vascular dissection or perforation, vascular aneurysm, vulnerable plaque, chronic total occlusion, claudication, anastomotic proliferation (for vein and artificial grafts), bile duct obstruction, ureter obstruction, tumor obstruction, and combinations thereof.

### Co-Solvent

As used herein, co-solvent refers to a solvent or solvent mixture capable of dissolving a polymer for forming the topcoat (topcoat polymer) and capable of dissolving, swelling or plasticizing a polymer for forming the basecoat (basecoat polymer) on a device. A co-solvent described herein provides the opportunity for the chain of a topcoat polymer to entangle with the top layer of the dissolved, swelled, or plasticized basecoat before drying. A co-solvent can be a single solvent or a mixture of solvents. In the mixture of solvents, the solvents shall be mutually miscible or substantially miscible. In some embodiments, the co-solvent can be a mixture of a solvent for a topcoat polymer and a solvent for a basecoat polymer.

In some embodiments, the polymer for forming the topcoat is a poly(ester amide) (PEA). Solvents for a PEA polymer include, but are not limited to, for example, CH₂Cl₂, chloroform, dimethyl formamide (DMF), dimethyl acetamide (DMAc), dimethyl sulfoxide (DMSO), or combinations of these. In some embodiments, the solvent can be alcohols (e.g., methanol, ethanol, n-propanol, isopropanol, 1-butanol, 1,3-propan-di-ol, 1,4-butan-di-ol), cyclohexanone, trichloroethane, tetrachloroethane, acetone, tetrahydrofuran (THF), dioxane, toluene, ethyl acetate, methyl ethyl ketone (MEK), acetonitrile, or combinations of these. In some embodiments, solvents for PEA can include dioxane and cyclohexanone, which can gel the PEA polymer, but don't dissolve it. In some embodiments, it is possible that these solvents in combination with a true solvent could solubilize PEA.

In some embodiments, the polymer for forming the basecoat can be a fluoropolymer. The term “fluoropolymer” refers to any polymers or copolymers of a fluorinated olefin. Examples of the fluoropolymer include SOLEF® polymers such as PVDF-HFP. Solvents for the fluoropolymer are well known in the art.

In some embodiments, the co-solvent can be a mixture of two solvents. The co-solvent can have different ratios of the solvents for the topcoat polymer to the basecoat polymer. For example, a co-solvent can be a mixture of DMAc and methanol. The ratio of DMAc to methanol can be between about 10:90 and about 90:10, preferably about 50:50. In some embodiments, alcohols such as ethanol or 1,4-butane-di-ol can be used in place of the methanol. Formulations with longer chain alcohols would necessitate smaller DMAc:alcohol ratios. For example, the co-solvent can be a mixture of DMAc and ethanol having a ratio of DMAc:ethanol of about 40:60 or a mixture of DMAc and 1,4-butan-di-ol having a ratio of DMAc:1,4-butan-di-ol of about 30:70. In some embodiments, cyclohexanone can be used in place of DMAc. PEA has limited solubility in cyclohexanone. The ratio of cyclohexanone to alcohol shall be between about 15:85 and about 30:70. A longer chain alcohol can also be used with cyclohexanone. The same trend of ratio variation in the DMAc:alcohol system also applies to cyclohexanone:alcohol. For example, a co-solvent of cyclohexanone and methanol can have a ratio of cyclohexanone:methanol of about 30:70 while a co-solvent of cyclohexanone and 1,4-butane-di-ol shall have a ratio of cyclohexanone:1,4-butane-di-ol of about 15:85.

The term poly(ester amide) includes any polymer that has at least an ester grouping and at least an amide grouping in its backbone. Some exemplary PEA polymers include three building blocks: an amino acid, a diol, and a diacid. The diacid can be, for example, a C2 to C12 diacid (e.g., aliphatic diacid with or without unsaturation or aromatic diacid). The diol can be, for example, a C2 to C12 diol, which can be a straight diol or branch diol with or without unsaturation. The amino acid can be, for example, glycine, valine, alanine, leucine, isoleucine, and/or phenyl alanine. An optional second amino acid can be included, which could include lysine, tyrosine, glutamic acid, or cysteine. The second amino acid can also contain a side group for attaching to a bioactive agent (e.g., pharmacologically active compound(s)) or property modifier(s). Some exemplary methods of making PEA are described in U.S. Pat. No. 6,503,538 B1. In some embodiments, the PEA polymer can be synthesized according to Scheme I:

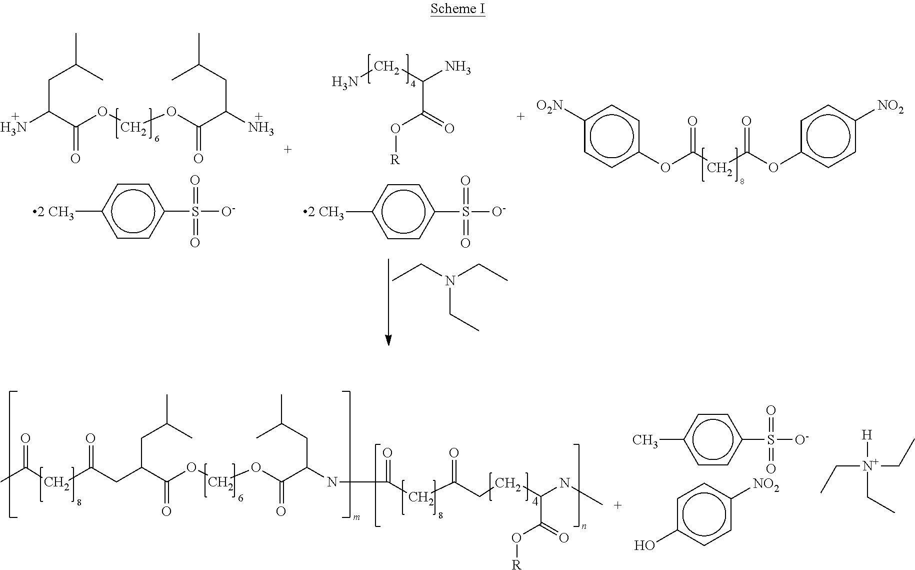

In some embodiments, the term poly(ester amide) can specifically exclude any polymer listed above.

### Basecoat

The method described herein can be used to form a topcoat on any basecoat, which can be also referred to as a substrate coating. The substrate coating can include one or more biocompatible polymer(s). The biocompatible polymer can be biodegradable (both bioerodable or bioabsorbable) or nondegradable. Representative biocompatible polymers include, but are not limited to, poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyurethanes, polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. PEO/PLA), polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as HEMA, hydroxypropyl methacrylate (HPMA), hydroxypropylmethacrylamide, PEG acrylate (PEGA), PEG methacrylate, 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, collagen, dextran, dextrin, fragments and derivatives of hyaluronic acid, heparin, fragments and derivatives of heparin, glycosamino glycan (GAG), GAG derivatives, polysaccharide, elastin, chitosan, alginate, or combinations thereof. In some embodiments, the substrate coating described herein can exclude any one of the aforementioned polymers.

As used herein, the terms poly(D,L-lactide), poly(L-lactide), poly(D,L-lactide-co-glycolide), and poly(L-lactide-co-glycolide) can be used interchangeably with the terms poly(D,L-lactic acid), poly(L-lactic acid), poly(D,L-lactic acid-co-glycolic acid), or poly(L-lactic acid-co-glycolic acid), respectively.

In some embodiments, the substrate coating or basecoat preferably includes a fluoropolymer such as a SOLEF™ polymer (e.g., PVDF-HFP).

In some embodiments, the substrate coating can further include a biobeneficial material. The biobeneficial material can be polymeric or non-polymeric. The biobeneficial material is preferably substantially non-toxic, non-antigenic and non-immunogenic. A biobeneficial material is one that enhances the biocompatibility of a device by being non-fouling, hemocompatible, actively non-thrombogenic, or anti-inflammatory, all without depending on the release of a pharmaceutically active agent.

Representative biobeneficial materials include, but are not limited to, polyethers such as poly(ethylene glycol), copoly(ether-esters) (e.g. PEO/PLA), polyalkylene oxides such as poly(ethylene oxide), polypropylene oxide), poly(ether ester), polyalkylene oxalates, polyphosphazenes, phosphoryl choline, choline, poly(aspirin), polymers and co-polymers of hydroxyl bearing monomers such as hydroxyethyl methacrylate (HEMA), hydroxypropyl methacrylate (HPMA), hydroxypropylmethacrylamide, poly (ethylene glycol) acrylate (PEGA), PEG methacrylate, 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as fibrin, fibrinogen, cellulose, starch, collagen, dextran, dextrin, hyaluronic acid, fragments and derivatives of hyaluronic acid, heparin, fragments and derivatives of heparin, glycosamino glycan (GAG), GAG derivatives, polysaccharide, elastin, chitosan, alginate, silicones, POLYACTIVE™, and combinations thereof. In some embodiments, the substrate coating can exclude any one of the aforementioned polymers.

The term POLYACTIVE™ refers to a block copolymer having flexible poly(ethylene glycol) and poly(butylene terephthalate) blocks (PEGT/PBT). POLYACTIVE™ is intended to include AB, ABA, BAB copolymers having such segments of PEG and PBT (e.g., poly(ethylene glycol)-block-poly(butyleneterephthalate)-block poly(ethylene glycol) (PEG-PBT-PEG).

In a preferred embodiment, the biobeneficial material can be a polyether such as poly (ethylene glycol) (PEG) or polyalkylene oxide.

### Bioactive Agents

In some embodiments, the coating having the features described herein can include one or more bioactive agents. The bioactive agents can be any bioactive agent that is therapeutic, prophylactic, or diagnostic. These agents can have anti-proliferative or anti-inflammatory properties or can have other properties such as antineoplastic, antiplatelet, anti-coagulant, anti-fibrin, antithrombonic, antimitotic, antibiotic, antiallergic, and antioxidant properties. These agents can be cystostatic agents, agents that promote the healing of the endothelium such as NO releasing or generating agents, agents that attract endothelial progenitor cells, or agents that promote the attachment, migration and proliferation of endothelial cells (e.g., natriuretic peptide such as CNP, ANP or BNP peptide or an RGD or cRGD peptide), while quenching smooth muscle cell proliferation. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules that bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of anti-proliferative agents include rapamycin and its functional or structural derivatives, 40-O-(2-hydroxy)ethyl-rapamycin (everolimus), and its functional or structural derivatives, paclitaxel and its functional and structural derivatives. Examples of rapamycin derivatives include methyl rapamycin (ABT-578), 40-O-(3-hydroxy)propyl-rapamycin, 40-O-[2-(2-hydroxy)ethoxy]ethyl-rapamycin, and 40-O-tetrazole-rapamycin. Examples of paclitaxel derivatives include docetaxel. Examples of antineoplastics and/or antimitotics include methotrexate, azathioprine, vincristine, vinblastine, fluorouracil, doxorubicin hydrochloride (e.g. ADRIAMYCIN® from Pharmacia & Upjohn, Peapack N.J.), and mitomycin (e.g. MUTAMYCIN® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as ANGIOMAX™ (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name MEVACOR from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of anti-inflammatory agents including steroidal and non-steroidal anti-inflammatory agents include tacrolimus, dexamethasone, clobetasol, combinations thereof. Examples of such cytostatic substance include angiopeptin, angiotensin converting enzyme inhibitors such as captopril (e.g. CAPOTEN® and CAPOZIDE® from Bristol-Myers Squibb Co., Stamford, Conn.), cilazapril or lisinopril (e.g. PRINIVIL® and PRINZIDE® from Merck & Co., Inc., Whitehouse Station, N.J.). An example of an antiallergic agent is permirolast potassium. Other therapeutic substances or agents that may be appropriate include alpha-interferon, pimecrolimus, imatinib mesylate, midostaurin, bioactive RGD, and genetically engineered endothelial cells. The foregoing substances can also be used in the form of prodrugs or co-drugs thereof. The foregoing substances also include metabolites thereof and/or prodrugs of the metabolites. The foregoing substances are listed by way of example and are not meant to be limiting. Other active agents that are currently available or that may be developed in the future are equally applicable.

The dosage or concentration of the bioactive agent required to produce a favorable therapeutic effect should be less than the level at which the bioactive agent produces toxic effects and greater than the level at which non-therapeutic results are obtained. The dosage or concentration of the bioactive agent can depend upon factors such as the particular circumstances of the patient, the nature of the trauma, the nature of the therapy desired, the time over which the ingredient administered resides at the vascular site, and if other active agents are employed, the nature and type of the substance or combination of substances. Therapeutic effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art.

### Examples of Medical Device

As used herein, a medical device may be any suitable medical substrate that can be implanted in a human or veterinary patient. Examples of such medical devices include self-expandable stents, balloon-expandable stents, stent-grafts, grafts (e.g., aortic grafts), heart valve prostheses, cerebrospinal fluid shunts, pacemaker electrodes, catheters, and endocardial leads (e.g., FINELINE™ and ENDOTAK™, available from Guidant Corporation, Santa Clara, Calif.), anastomotic devices and connectors, orthopedic implants such as screws, spinal implants, and electro-stimulatory devices. The underlying structure of the device can be of virtually any design. The device can be made of a metallic material or an alloy such as, but not limited to, cobalt chromium alloy (ELGILOY™), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable (e.g., bioabsorbable stent) or biostable polymers could also be used with the embodiments of the present invention.

### Method of Use

Preferably, the medical device is a stent. The stent described herein is useful for a variety of medical procedures, including, by way of example, treatment of obstructions caused by tumors in bile ducts, esophagus, trachea/bronchi and other biological passageways. A stent having the above-described coating is particularly useful for treating diseased regions of blood vessels caused by lipid deposition, monocyte or macrophage infiltration, or dysfunctional endothelium or a combination thereof, or occluded regions of blood vessels caused by abnormal or inappropriate migration and proliferation of smooth muscle cells, thrombosis, and restenosis. Stents may be placed in a wide array of blood vessels, both arteries and veins. Representative examples of sites include the iliac, renal, carotid and coronary arteries.

For implantation of a stent, an angiogram is first performed to determine the appropriate positioning for stent therapy. An angiogram is typically accomplished by injecting a radiopaque contrasting agent through a catheter inserted into an artery or vein as an x-ray is taken. A guidewire is then advanced through the lesion or proposed site of treatment. Over the guidewire is passed a delivery catheter that allows a stent in its collapsed configuration to be inserted into the passageway. The delivery catheter is inserted either percutaneously or by surgery into the femoral artery, radial artery, brachial artery, femoral vein, or brachial vein, and advanced into the appropriate blood vessel by steering the catheter through the vascular system under fluoroscopic guidance. A stent having the above-described coating may then be expanded at the desired area of treatment. A post-insertion angiogram may also be utilized to confirm appropriate positioning.

### EXAMPLES

### Example 1

### Improved re-EC Kinetics of Poly(Ester Amide)

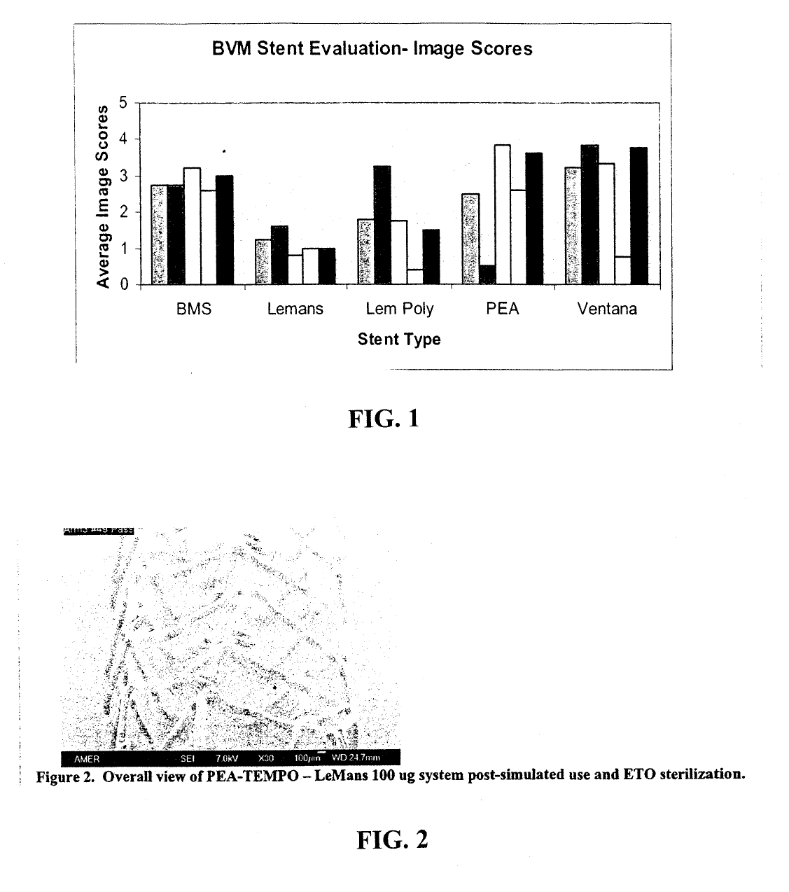

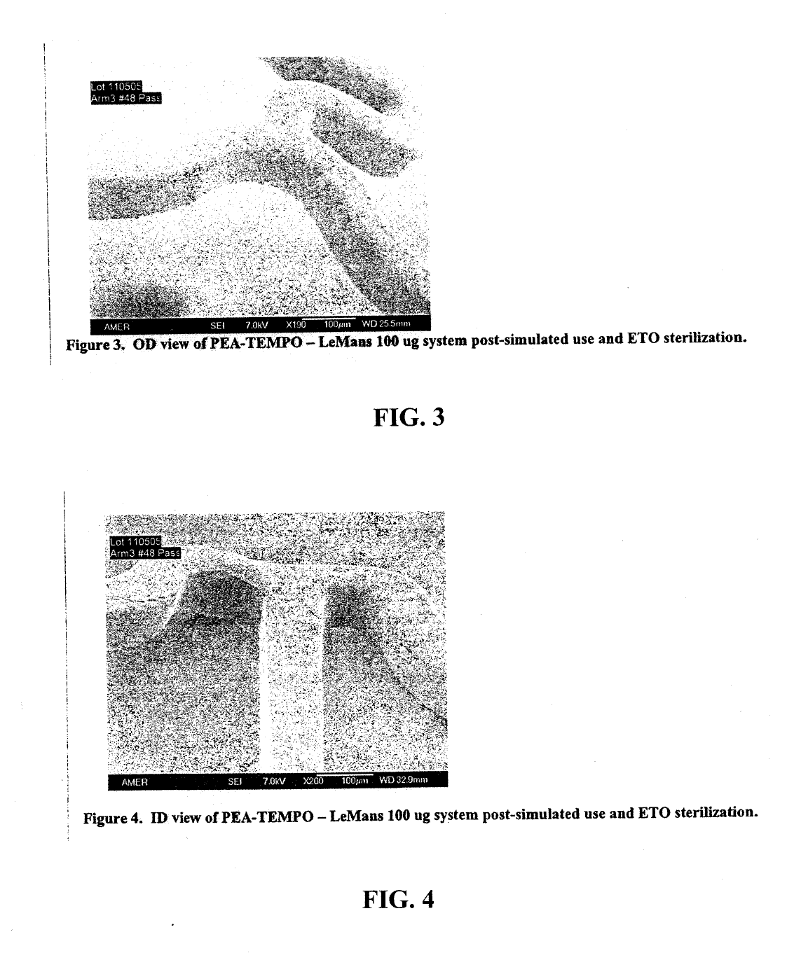

PEA-TEMPO coated stents (3.0×12 mm small VISION™ stents, available from Guidant Corporation, Santa Clara, Calif., coated with 736 μg PEA-TEMPO) and stents coated with PEA-TEMPO/everolimus (3.0×12 mm small VISION™ stents) (VENTANA™) coated stents (D:P1:6, 100 μg/cm² drug dose with a 400 μg PEA-TEMPO topcoat) were implanted in a bioengineered vessel to benchmark re-endothelialization at the 14 day time point. The PEA-TEMPO and PEA-TEMPO/everolimus coated stents were compared with bare metal stent (BMS) (VISION™) and Lemans stents (stents coated with a PBMA primer, a reservoir layer, and a SOLEF™ topcoat) (3.0×12 mm small VISION™ stents, with a 100 μg/cm² dose, drug:polymer (D:P)=1:4.9). The stented vessels were stained with bisbenzimide (BBI), cut in half longitudinally, and imaged with a 10× objective. Images were assessed according to a scoring system (0—no cells or protein; 1—no cells; some protein; 2—some interspersed cells; 3—localized cell density in some areas; 4—consistent cell density covering most of the stent; 5—highest cell density, masking stent) and averaged across the sample. The PEA-TEMPO and VENTANA™ stents were found to have endothelial cell coverage similar to BMS and greater than Lemans polymer coated stents, indicating a prohealing potential. The results are summarized in FIG. 1. The one low outlier for PEA is due to a bioreactor failure and should be discounted. Other variability within the data (e.g., low PEA-TEMPO/everolimus outlier, low and high Lemans polymer) may be due to stent deployment differences, stent malaposition, or inconsistent cell linings at time zero (t₀).

### Example 2

### Mechanical Integrity of ETO Sterilized PEA-TEMPO Topcoated Lemans Stents

The following example illustrates how PEA-TEMPO can be used as a topcoat on the Lemans platform (100 μg/cm²) while not compromising the mechanical integrity of the stents.

Small 12 mm VISION™ stents (available from Guidant Corporation, Santa Clara, Calif.) were spray-coated with 51 μg PBMA primer and 378 μg SOLEF/everolimus, D:P1:4.9 with a100 μg/cm² dose) (referred to as “LeMans stent”), and, then, 100 μg of PEA-TEMPO was spray coated on top of the LeMans stent. The PEA-TEMPO layer was coated from a 2 wt % solids in 200 proof ethanol solution.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

We claim:

1. A method comprising:
priming a basecoat on a medical device with a blank solvent spray, and
applying a topcoat formulation to the primed basecoat,
wherein the interfacial compatibility between the topcoat and the basecoat is improved.

2. The method of claim 1, wherein the basecoat comprises a fluoropolymer, and
wherein the topcoat comprises a PEA polymer.

3. The method of claim 2, wherein the fluoropolymer is PVDF-HFP.

4. The method of claim 2, wherein the basecoat comprises a bioactive agent.

5. The method of claim 4, wherein the bioactive agent comprises a component selected from paclitaxel, docetaxel, estradiol, nitric oxide donors, super oxide dismutases, super oxide dismutases mimics, 4-amino-2,2,6,6-tetramethylpiperidine-1-oxyl (4-amino-TEMPO), biolimus, tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N-1-tetrazolyl)-rapamycin (ABT-578), clobetasol, pimecrolimus, imatinib mesylate, midostaurin, prodrugs thereof, co-drugs thereof, or combinations of these.

6. The method of claim 4, wherein the medical device is a stent.

7. The method of claim 4, wherein the medical device is a bioabsorbable stent.

8. A method comprising:
exposing a basecoat of a medical device to a solvent-rich atmosphere comprising a solvent capable of plasticizing or absorbing into the top layer of the basecoat, and
applying a topcoat formulation to the basecoat,
wherein the topcoat and the basecoat have an improved interfacial compatibility.

9. The method of claim 8, wherein the basecoat comprises a fluoropolymer, and wherein the topcoat comprises a PEA polymer.

10. The method of claim 9, wherein the fluoropolymer is PVDF-HFP.

11. The method of claim 9, wherein the basecoat comprises a bioactive agent.

12. The method of claim 11, wherein the bioactive agent comprises a component selected from paclitaxel, docetaxel, estradiol, nitric oxide donors, super oxide dismutases, super oxide dismutases mimics, 4-amino-2,2,6,6-tetramethylpiperidine-1-oxyl (4-amino-TEMPO), biolimus, tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N-1-tetrazolyl)-rapamycin (ABT-578), clobetasol, pimecrolimus, imatinib mesylate, midostaurin, prodrugs thereof, co-drugs thereof, or combinations of these.

13. The method of claim 11, wherein the medical device is a stent.

14. The method of claim 11, wherein the medical device is a bioabsorbable stent.

15. A coating on a medical device formed according to the method of claim 3 comprising a basecoat portion and a topcoat portion with an improved interfacial compatibility between the topcoat and basecoat portions.

16. A coating on a medical device formed according to the method of claim 4 comprising a basecoat portion and a topcoat portion with an improved interfacial compatibility between the topcoat and basecoat portions.

17. A coating on a medical device formed according to the method of claim 6 comprising a basecoat portion and a topcoat portion with an improved interfacial compatibility between the topcoat and basecoat portions.

18. A coating on a medical device formed according to the method of claim 8 comprising a basecoat portion and a topcoat portion with an improved interfacial compatibility between the topcoat and basecoat portions.

19. A coating on a medical device formed according to the method of claim 10 comprising a basecoat portion and a topcoat portion with an improved interfacial compatibility between the topcoat and basecoat portions.

20. A coating on a medical device formed according to the method of claim 11 comprising a basecoat portion and a topcoat portion with an improved interfacial compatibility between the topcoat and basecoat portions.

21. A coating on a medical device formed according to the method of claim 13 comprising a basecoat portion and a topcoat portion with an improved interfacial compatibility between the topcoat and basecoat portions.

22. A method of treating a disorder in a patient comprising implanting in the patient a medical device with the coating of claim 18, wherein the disorder is at least one of atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, and combinations thereof.

23. The method of claim 2, wherein the basecoat comprises a bioactive agent provided that the bioactive agent is not actinomycin.
